(19) United States
(12) Patent Application Publication
(10) Pub. No.: US 2007/0282437 A1
(43) Pub. Date: Dec. 6, 2007

(54) Temporary Anti-Photophobic Ocular Sevice and Method for Production Thereof

(75) Inventors: Laurence Hermitte, Annecy Le Vieux (FR); Olivier Benoit, Epagny (FR); Gilles Bos, Plan les Ouates (CH)

Correspondence Address: YOUNG & THOMPSON, 745 SOUTH 23RD STREET, 2ND FLOOR, ARLINGTON, VA, 22202, US

(73) Assignee: XCELENS S.A., Geneve (CH)

(21) Appl. No.: 11/660,992

(22) PCT Filed: Aug. 18, 2005
(86) PCT No.: PCT/FR05/02101

§ 371 Date: May 21, 2007

(30) Foreign Application Priority Data
Sep. 3, 2004 (FR) 0409341

(51) Int. Cl. A61F 2/16

(52) U.S. Cl. 623/6.17

(57) ABSTRACT

The invention relates to an ocular device which permits the avoidance of the phenomenon of photophobia and the method for production thereof. The device comprises one or more chromophores which absorb light in the wavelengths between 400 and 575 nm (blue-green light) and which are dispersed, after the placement in or on the eye, in the surrounding body fluids in several weeks to several months depending on the concentration of the chromophores. The device is produced by conditioning of the non-colored ocular device in an aqueous solution of the chromophore(s) in the shade.

The invention relates to a temporary anti-photophobia ocular device and its process for preparation.

Photophobia corresponds to intolerance to light.

The individuals suffering from photophobia are very sensitive to strong luminosity and can be stricken with headaches. In extreme cases, the light can be irritating.

This is a symptom that is often associated with:
Physiological disorders: corneal inflammation, uveitis, keratitis, conjunctivitis, retinal detachment, disorders of the central nervous system.
Refractive surgery or cataract surgery.

At present, physicians, and particularly surgeons after a cataract operation, recommend that their patients who suffer from photophobia wear sunglasses.

One of the objects of this invention is to propose an alternative to wearing glasses after cataract surgery, which ensures better comfort to the patient without thereby reducing his viewing of colors and contrasts for the long term. Another object is to provide a device (contact lens, for example) that overcomes a photophobia of another origin.

In the first case, the invention consists of a temporarily colored implant so that the patient adapts more easily to the change in transmission capacities of the light between the natural crystalline lens—which has yellowed over time and which is eliminated during the surgery—and the newly implanted lens. The coloring agent that is integrated in the implant is characterized by filtration capacities of the light in the wavelengths between 400 and 575 nm. This coloring agent is gradually released, after the installation of the implant, in the surrounding fluids. The patient is thus protected against hypersensitivity to the light immediately after surgery and will recover bit by bit, thanks to a gradual increase of the percentage of transmission of the previously absorbed wavelengths, an optimum viewing of colors and contrasts; this aspect is all the more significant as the luminosity is increasingly sought by aged individuals and as good viewing of colors and contrasts is necessary for a good quality of life.

In the second case, the invention consists of a temporarily colored contact lens under the same conditions as above.

There are already permanently colored ocular devices for blocking blue-violet light, the Alcon AcrySof Natural implant or the Hoya AF1 (uy) implant. Their main objective is to protect the eye from the macular degeneration that highly energetic wavelengths can induce, even if this is subject to controversy. They filter only the short wavelengths, and this in a definitive way. However:
1. They irreversibly modify the viewing of colors and contrasts of the patient (reduction of the scotopic performance).
2. The photophobia and the phototoxicity are not only due to short wavelengths but also to blue-green light.

There is therefore provided, according to the invention, an ocular device that makes it possible to prevent the photophobia phenomenon, following in particular a surgical operation of the eye, whereby the ocular device contains one or more chromophores that absorb the light in wavelengths that are 400 to 575 nm and that can be dropped back, after implantation, into the surrounding fluids within several weeks to several months based on the chromophore concentration and the affinity of the chromophore for the constituent material of the device.

The ocular device is advantageously an intraocular lens made of hydrogel polymer (such as, for example, poly(hydroxylethylmethacrylate), the acrylic or methacrylic copolymers of which one of the monomers is advantageously hydroxyethylmethacrylate, the hydroxyethylmethacrylate/silicone matrices, the polymers with an N-vinylpyrrolidone base, etc.).

The chromophore or chromophores that are used are selected, of course, from among the non-toxic and biocompatible chromophores that can be extracted from the ocular device by dissolving or entrainment via the surrounding biological fluids. They absorb the light in wavelengths of 400 to 575 nm, and by way of preferred examples, it is possible to cite the riboflavin in aqueous solution that is less than or equal to 3% (m/v) and the fluorescein in aqueous solution that is less than or equal to 0.6% (m/v).

The chromophore or chromophores are set temporarily in the hydrogel polymer either by absorption/diffusion or by covalent or ionic association according to the nature of the chromophore concerned.

The invention also relates to the process for preparation of the ocular device, which comprises the stage that consists in placing a non-colored ocular device in an aqueous solution of the chromophore or chromophores, in the absence of light, whereby the impregnation period is based on the temperature.

The description will now be given in the form of nonlimiting examples.

In all of the examples, a hydroxyl polymer pellet with a thickness of 0.8 mm and a diameter of 10 mm is used as a starting pellet.

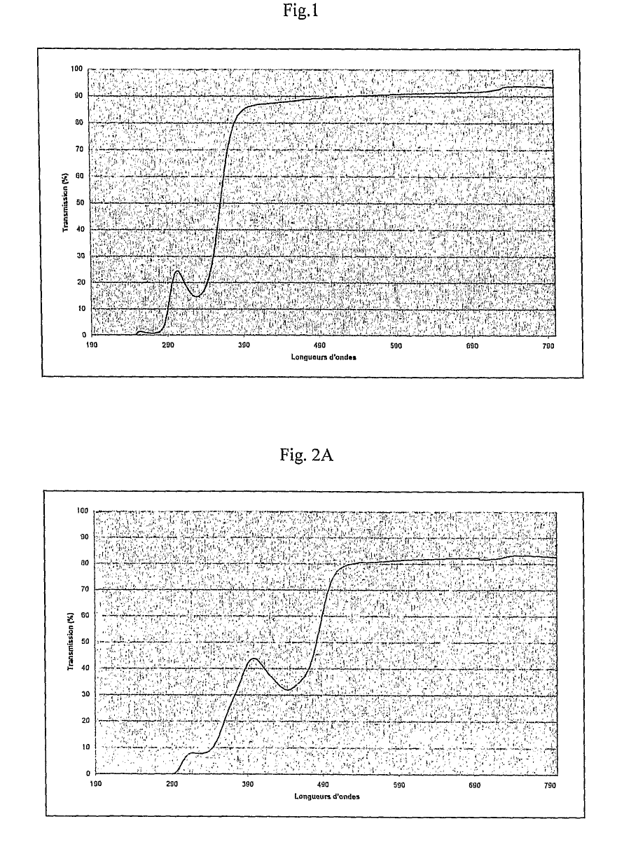

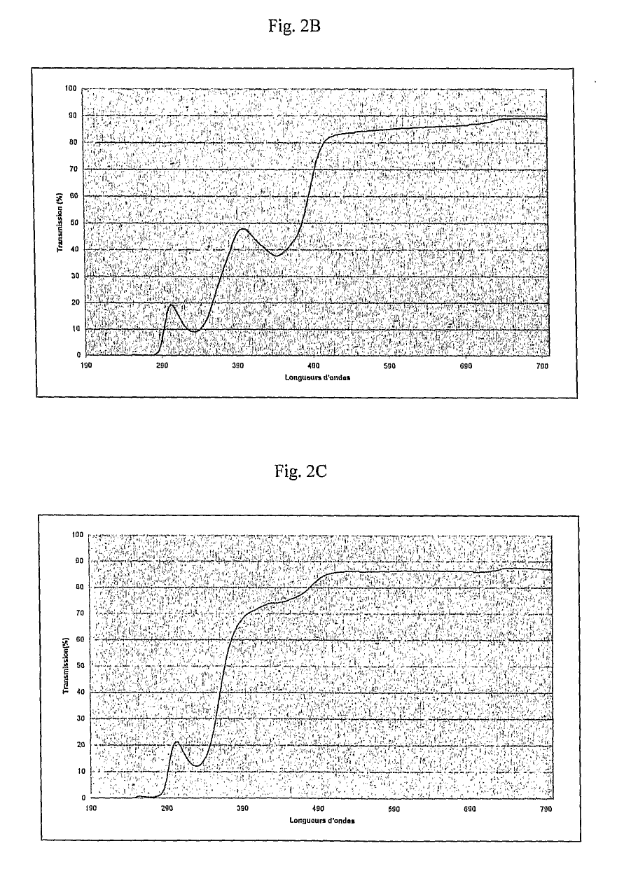

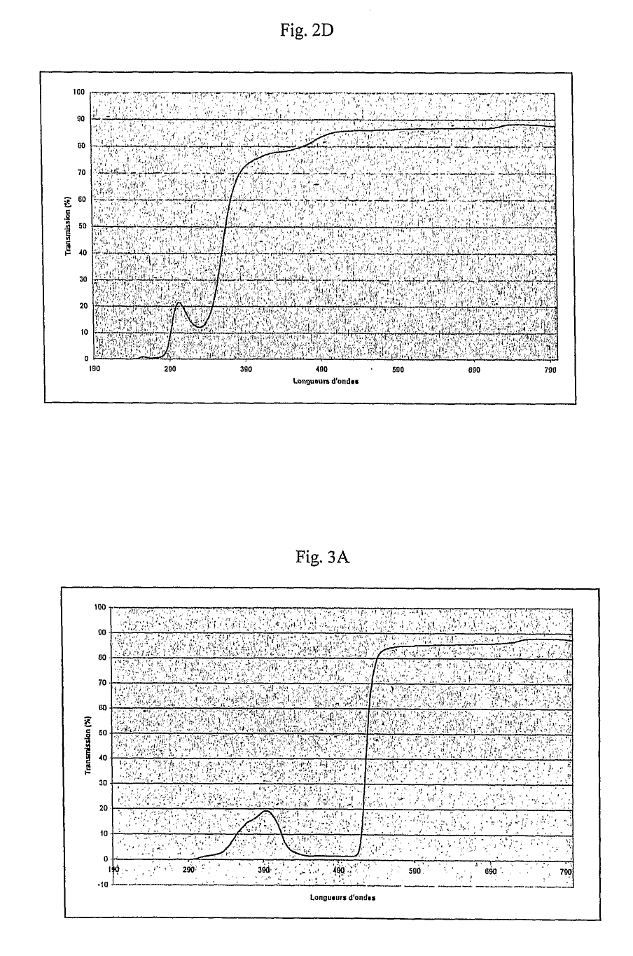

FIG. 1 is the transmission spectrum of a hydrogel polymer pellet before coloring, while FIGS. 2A to 2D are transmission spectra of a hydrogel polymer pellet that is colored with a 1% (m/v) riboflavin solution on days 0, 1, 30 and 60 of conditioning in a 0.9% sodium chloride solution.

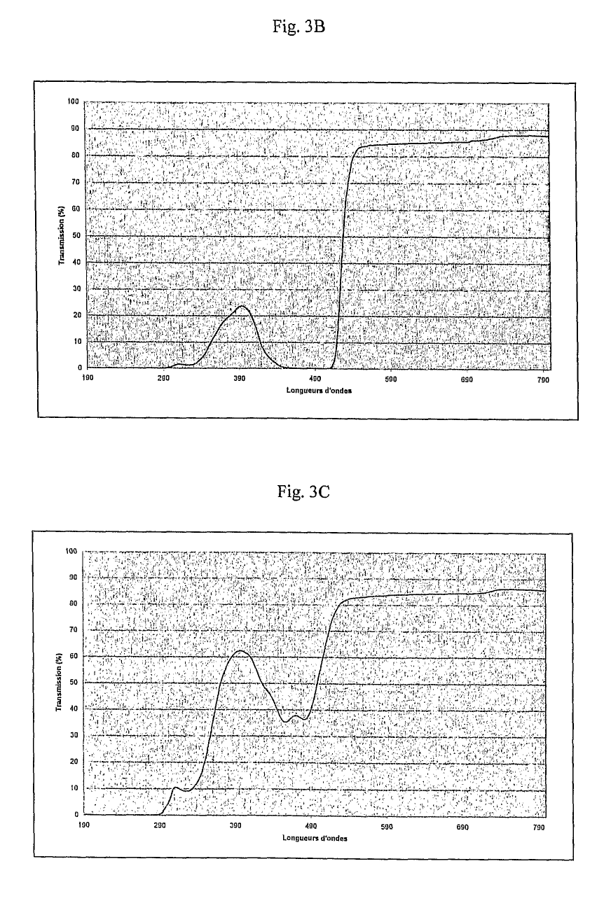

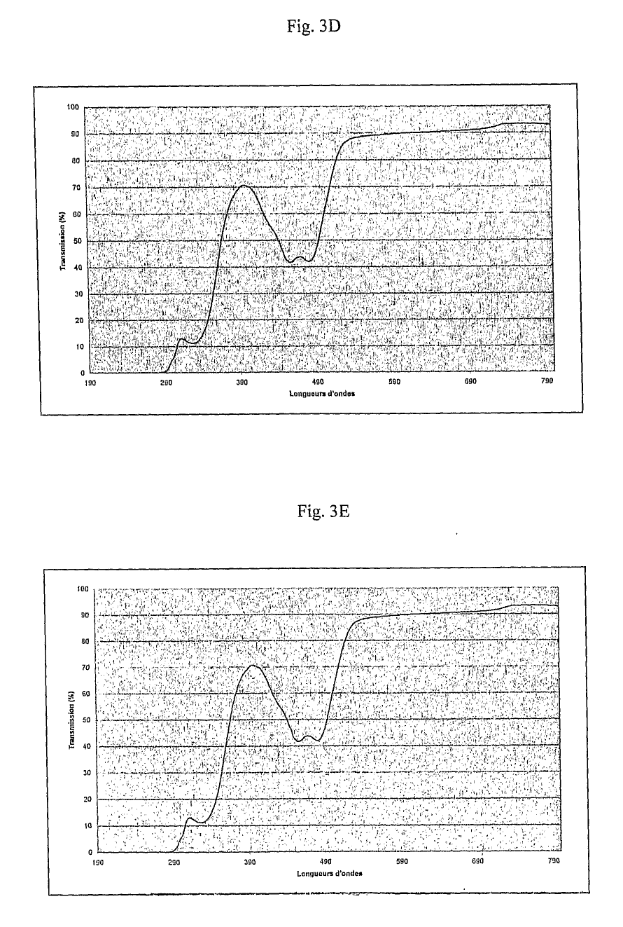

FIGS. 3A to 3E are transmission spectra of a hydrogel polymer pellet that is colored by a 0.6% (m/v) fluorescein solution on days 0, 1, 30, 60 and 90 for quenching in a 0.9% sodium chloride solution.

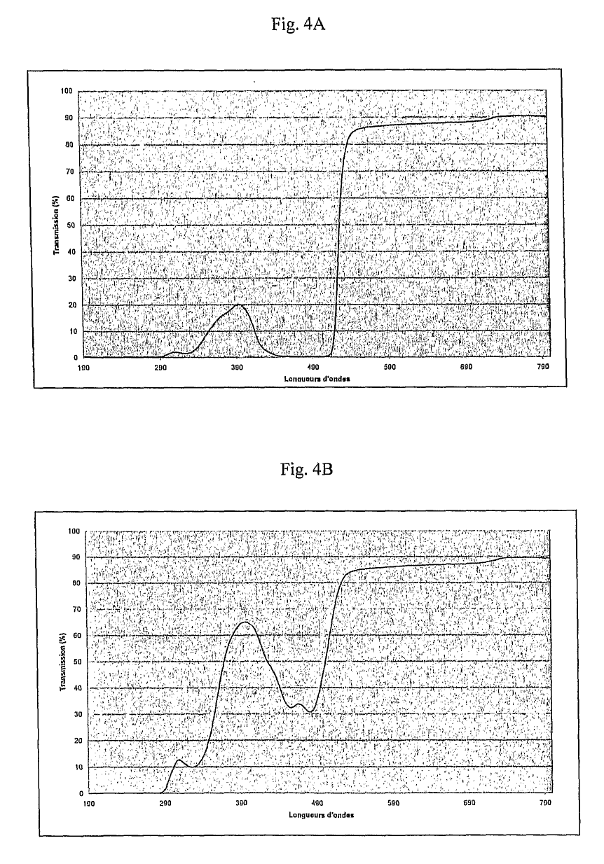

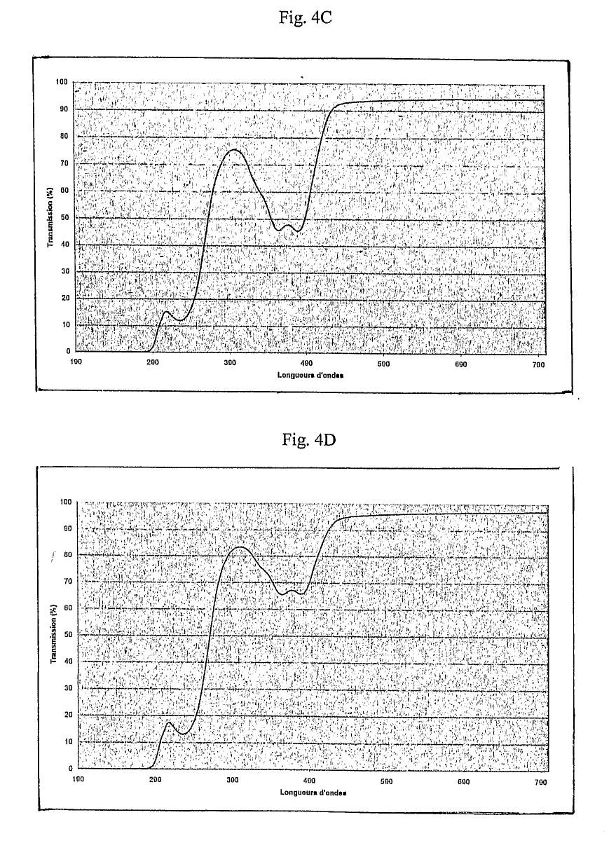

FIGS. 4A, 4B, 4C and 4D are transmission spectra of a hydrogel polymer pellet by a 0.1% (m/v) fluorescein solution on days 0, 15, 45 and 60 for quenching in a 0.9% sodium chloride solution.

### EXAMPLES (IN VITRO)

### Example 1

A 1% (m/v) riboflavin aqueous solution is prepared. A hydrogel polymer pellet (Benz 25UV) that has a hydration rate of 25% (m/v) is conditioned in the riboflavin solution for 7 days at ambient temperature and in the absence of light. The pellet before conditioning had the transmission spectrum that is shown in FIG. 1.

The colored pellet is then conditioned in a bottle containing 5 ml of 0.9% (m/v) sodium chloride aqueous solution (physiological serum) at ambient temperature and in the absence of light. The 0.9% NaCl solution is replaced every 3 days during the experiment.

FIG. 2A provides the transmission spectrum of the colored pellet after seven days of conditioning in the riboflavin solution, and a clear absorption of wavelengths between about 400 and 520 nm is noted, which makes it possible to significantly reduce the amount of light that is transmitted to the retina while not negating the scotopic efficiency.

FIGS. 2B, 2C and 2D correspond to the transmission spectra respectively after 1 day, 30 days and 60 days in the NaCl solution and show the elimination or progressive dropping back of the riboflavin, whereby the polymer gradually recovers its initial light transmission properties.

### Example 2

A 0.6% (m/v) fluorescein aqueous solution is prepared. A hydrogel polymer pellet, identical to the one that is used in Example 1, is conditioned in the fluorescein solution for 7 days at ambient temperature and in the absence of light. After 7 days, the pellet is conditioned in a bottle that contains 5 ml of a 0.9% (m/v) NaCl aqueous solution at ambient temperature and in the absence of light. The NaCl solution is replaced every 3 days during the experiment.

FIGS. 3A to 3E show the transmission spectra on days 0, 1, 30, 60 and 90 of conditioning in the NaCl solution. Initially, the absorption is greater than 80% for wavelengths of less than 520 nm. It is noted that the polymer recovers its initial capacities of light transmission bit by bit, more slowly, however, than in Example 1.

### Example 3

A 0.1% (m/v) fluorescein aqueous solution is prepared. A hydrogel polymer pellet, identical to the one that is used in the preceding examples, is conditioned in the fluorescein solution for 7 days at ambient temperature and in the absence of light. After 7 days, the pellet is conditioned in a bottle that contains 5 ml of a 0.9% (m/v) NaCl aqueous solution at ambient temperature and in the absence of light. The NaCl solution is replaced every 3 days during the experiment.

FIGS. 4A to 4D show the transmission spectra on days 0, 15, 45, and 60 of conditioning in the NaCl solution. It is noted that the transmission spectrum on day 0 is similar to the one that is obtained with a 0.6% fluorescein solution (Example 2), (transmission very slightly greater around 400 nm when the initial concentration is lower). The polymer recovers its initial transmission spectrum bit by bit: after 60 days of conditioning in NaCl, the percentage of transmission of the most filtered wavelengths through the chromophore is more than 65%. The polymer recovers its initial transmission spectrum bit by bit; after 60 days of conditioning in NaCl, the percentage of transmission of the most filtered wavelengths through the chromophore is more than 65%.

1. Ocular device that makes it possible to prevent the photophobia phenomenon, characterized in that the device contains one or more chromophores that absorb the light in the wavelengths that are between 400 and 575 nm and that can be dropped back after being put in or on the eye in surrounding fluids within several weeks to several months based on the concentration of chromophores and the affinity of the chromophore for the constituent material of the device.

2. Ocular device according to claim 1, wherein it is an intraocular lens made of hydrogel polymer.

3. Ocular device according to claim 1, wherein it is colored by riboflavin or fluorescein.

4. Process for preparation of an ocular device according to claim 1, wherein it consists in placing a non-colored ocular device in an aqueous solution of the chromophore or chromophores and in the absence of light.

5. Process according to claim 4, wherein the chromophore solution is an aqueous solution of riboflavin that is less than or equal to 3% (m/v).

6. Ocular process according to claim 4, wherein the chromophore solution is an aqueous solution of fluorescein that is less than or equal to 0.6% (m/v).

7. Ocular device according to claim 2, wherein it is colored by riboflavin or fluorescein.

8. Process for preparation of an ocular device according to claim 2, wherein it consists in placing a non-colored ocular device in an aqueous solution of the chromophore or chromophores and in the absence of light.

9. Process for preparation of an ocular device according to claim 3, wherein it consists in placing a non-colored ocular device in an aqueous solution of the chromophore or chromophores and in the absence of light.
